(19) United States
(12) Patent Application Publication
(10) Pub. No.: US 2003/0073904 A1
(43) Pub. Date: Apr. 17, 2003

(54) Ultrasonic medical treatment equipment and ultrasonic diagnostic equipment

(75) Inventors: Tadashi Moriya, Yokohama-shi (JP); Yoshikatsu Tanahashi, Sendai-shi (JP); Rikio Honda, Sendai-shi (JP)

Correspondence Address: STAAS & HALSEY LLP, 700 11TH STREET, NW, SUITE 500, WASHINGTON, DC, 20001, US

(73) Assignee: Hondaseiki Corporation, Sendai (JP)

(21) Appl. No.: 10/236,941

(22) Filed: Sep. 9, 2002

(30) Foreign Application Priority Data
Oct. 15, 2001 (JP) 2001-358506

(51) Int. Cl. A61B 8/00

(52) U.S. Cl. 600/439

(57) ABSTRACT

A transmission unit to transmit an ultrasonic wave into a body and a medical treatment/diagnostic apparatus having the same. The transmission unit includes a fiber and a protective layer on a periphery of the fiber to prevent breaking due to surface cracks in the fiber during bending. Thus, the transmission unit can be inserted into the body through curved passages such as the urinary tract. The transmission units may be bundled to increase a power of the ultrasonic wave.

### CROSS-REFERENCE TO RELATED APPLICATIONS

This application claims the benefit of Japanese Application No. 2001-358506, filed Oct. 15, 2001, in the Japanese Patent Office, the disclosure of which is incorporated herein by reference.

### BACKGROUND OF THE INVENTION

Ultrasonic medical diagnostic and treatment equipment is widely used in the medical field in general and in the field of urology in particular. One example of ultrasonic diagnostic equipment creates an image of the inside of a body without entering the body. In the example of ultrasonic endoscopy, a vibrator is inserted into the human body to create the image. Furthermore, medical treatment equipment irradiates a powerful ultrasonic wave (shock wave) from the outside of the human body towards the affected part to thereby destroy a calculus or other abnormality. Ultrasonic medical treatment equipment may also irradiate an ultrasonic wave at a prostate gland using an array-type vibrator or with an ultrasonic endoscope, both of which are inserted into the rectum.

A problem with the known internal ultrasonic diagnostic equipment is that in order to fit into the body, the vibrator cannot be enlarged, and therefore the transmitting power of the ultrasonic waves cannot be increased. A problem with the known external ultrasonic generators is that skin, the air in the intestines, or bone may interfere with the signal before reaching the location of treatment. This problem even exists in the array-type equipment for rectal use.

One specific example of an ultrasonic wave applicator which generates a powerful ultrasonic wave outside of the human body for transmission through a transmission conduit into the brain is disclosed by B. J. Jarosz and D. Kaytar. Ultrasonic Wave Guide Applicator Arrays for Interstitial Heating, A Model Study, IEEE Trans. on Ultrasonics and Frequency Control, Vol. 45, No.3 (May 1998), pp.806-814. However, this reference discloses a thick and inflexible transmission conduit, therefore, it is difficult to use this apparatus in conjunction with an endoscope.

The use of quartz fiber has been disclosed in Japanese Patent Application No. 11-271454, however, this reference does not disclose sufficient flexibility for use in the human body. JP 58-29454 to Kokai discloses a flexible transmission conduit using water as a transmission medium, however, it is difficult for this apparatus to transmit a supersonic wave of a frequency of 1 MHz or more.

Thus, the known methods lack flexibility, power, or suffer from high attenuation of the ultrasonic wave. Furthermore, these designs are not flexible enough to reach affected areas, such as a cancer growth, which are located deep within the body. If an ultrasonic wave is applied from outside the body, a detailed image cannot be created due to interference. Furthermore, injury may occur to areas between the treatment equipment and the affected part.

### SUMMARY OF THE INVENTION

Accordingly, it is one potential aspect of the present invention to provide a flexible transmission conduit which can transmit an ultrasonic wave of a frequency of 1 MHz or more.

It is another potential aspect of the present invention to provide ultrasonic diagnostic equipment which can be used with an endoscope by using a transmission conduit having sufficient flexibility to be used inside the body. The transmission conduit is thin and flexible, to thereby accelerate diagnosis and treatment.

It is still another potential aspect of the present invention to provide ultrasonic treatment equipment which can directly apply a powerful ultrasonic wave to the affected part, with an ultrasonic transmitter which is located outside of the human body.

It is a further potential aspect of the present invention to overcome the above-mentioned problems of known ultrasonic diagnostic and treatment equipment.

Additional aspects and advantages of the invention will be set forth in part in the description which follows and, in part, will be obvious from the description, or may be learned by practice of the invention.

The above and other aspects and advantages of the present invention are achieved by providing an apparatus to transmit an ultrasonic wave with a transmission unit using a fiber covered with a protective layer which allows for bending without being easily damaged. An ultrasonic generating-receiving part and probe are located outside of the human body, and the ultrasound wave is transmitted to a transmission-and-reception part by a transmission unit which is inside the body.

### BRIEF DESCRIPTION OF THE DRAWINGS

These and other aspects and advantages of the invention will become apparent and more readily appreciated from the following description of the preferred embodiments, taken in conjunction with the accompanying drawings of which:

FIG. 1 is a schematic diagram showing the ultrasonic medical treatment/diagnostic equipment according to a first embodiment of the present invention;

FIG. 2 is a perspective view showing a transmission conduit according to the first embodiment of the present invention;

FIG. 3 is a view showing the transmission conduit as used during an examination according to the present invention;

FIG. 4 is a perspective view of a transmission unit according to a second embodiment of the present invention;

FIG. 5 is a schematic diagram showing the ultrasonic medical treatment/diagnostic equipment using the transmission conduit of FIG. 4;

FIG. 6 illustrates a third embodiment of the present invention;

FIG. 7 shows a transmission conduit according to a fourth embodiment the present invention; and

FIG. 8 is a schematic view showing the ultrasonic treatment/diagnostic equipment according to a fifth embodiment of the present invention.

### DETAILED DESCRIPTION OF THE PREFERRED EMBODIMENTS

Reference will now be made in detail to the present preferred embodiments of the present invention, examples of which are illustrated in the accompanying drawings, wherein like reference numerals refer to like elements throughout.

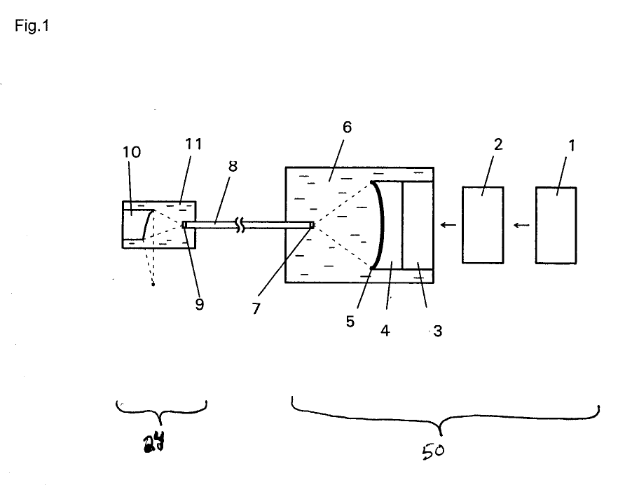

FIG. 1 illustrates a first embodiment of the present invention. An ultrasonic signal from generating circuit 1 is amplified by electric power amplifier 2, and is sent to a vibrator 3. A sound lens 4 having an adjustment layer 5 (made of polyethylene) converges the ultrasonic wave, which travels through a liquid 6, such as water or silicone oil. The generating circuit 1, power amplifier 2, vibrator 3 and sound lens 4 form a generating part 50, located outside the body.

The converged ultrasonic wave is received at adjustment layer 7, at the end of a transmission conduit 8, which has another adjustment layer 9, on an opposite end from adjustment layer 7. A tip part 24, forming a transmission-reception unit, is inserted into the body and includes a sound mirror 10, which reflects the ultrasonic wave sent through the transmission conduit 8, and a liquid 11. Thus, in this example, the tip part 24 serves as a medical treatment/diagnostic part. The sound mirror 10 has an elliptical surface of a parabolic shape, and a diameter of about 2 mm, or less, thereby allowing for insertion into the urinary tract.

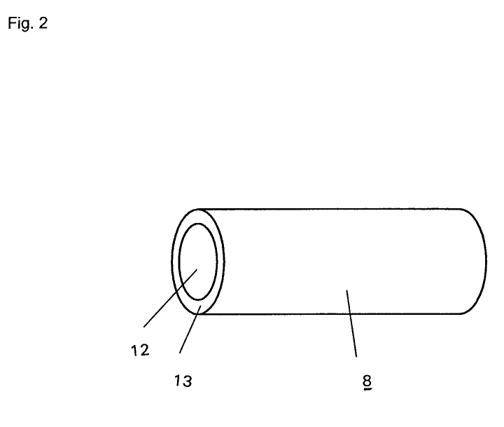

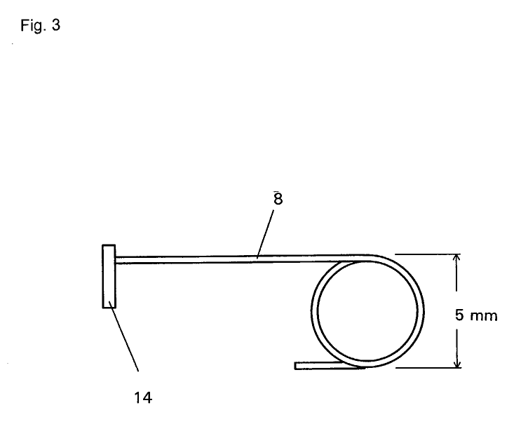

FIG. 2 is a perspective view of transmission conduit 8. The transmission conduit 8 includes a protective layer 13 on the perimeter thereof formed of carbon or aluminum, and a quartz fiber 12. Since the quartz fiber 12 is made by melting and extending silica glass, small cracks will form on the surface, thereby concentrating stresses when the quartz fiber 12 moves, causing cracking and breaking. The protective layer 13 prevents such breakage of the quartz fiber 12. As shown in FIG. 3, if aluminum is used as the protective layer 13, the transmission conduit 8 can be bent to a diameter of 5 mm. Moreover, if carbon is used as protective layer 13, the conduit 8 can be bent to a diameter of 30 mm. Thus, the material forming the protective layer 13 can be chosen according to the particular use.

The diagnosis and treatment using the apparatus according to the first embodiment will be now be discussed, using the example of treating a pelvic or renal neoplasm. The diameter of the tip part 24 is about 2 mm and is inserted to reach the neoplasm through the urethra by using a working channel of an optical endoscope that is previously inserted. The optical endoscope is then used to check the position of the tip part 24. When the tip part 24 is suitably positioned near the affected area, an ultrasonic electrical signal is generated by the generating circuit 1. The ultrasonic electrical signal is amplified by the electric power amplifier 2, and the vibrator 3 receives the amplified signal and generates an ultrasonic wave. The ultrasonic wave is converged by the sound lens 4 and connects a focus to the adjustment layer 7, and the ultrasonic wave is transmitted through the quartz fiber 12, and reaches the adjustment layer 9. The ultrasonic wave is irradiated through the sound mirror 10, and is then applied to the neoplasm, without inflicting damage on the kidney itself. The energy of the ultrasonic wave then destroys the neoplasm. Due to the use of protective layer 13, the fiber 12 of transmission conduit 8 can bend easily, thereby allowing the tip part 24 to be placed close to the affected area without the risk of breaking or cracking due to surface cracks.

FIG. 3 illustrates a curve examination using the transmission conduit 8 having a dispatch element 14. A loop with a diameter of 5 mm is formed, and a thickness of the protective layer 13 is 125 micrometers, or may be anywhere in a range of 100 to 200 micrometers. A length of the transmission conduit 8 is approximately 600 mm, but may be as long as 1000 mm. Under these conditions, the characteristics of the ultrasonic wave generated by the vibrator 3 are as follows. The transmission conduit 8 is spread by L (0 1) of the Pochhammer-Chree wave, L (0 2), and L (0 3) mode by low loss, and the energy which fully destroys the calculus which exists in the kidney can be transmitted. Although this embodiment uses a carbon layer as the protective layer 13, a protective layer 13 which forms a carbon nanotube using alumina system inorganic binder may also be used. The diameter of the nanotube may be approximately 10 nm. However, other sizes of carbon nanotube may be used. The tensile strength of a carbon nanotube is several times that of steel, and the nanotube also prevents breakage of the quartz fiber 12.

Aluminum having a thickness of 1-3 microns can also be used as the protective layer 13. The aluminum protective layer 13 is formed by carrying out vacuum evaporation of the aluminum to the quartz fiber 12. The protective layer 13 may also be formed by plating, after forming an electric conduction layer. Still further, as the protective layer 13, non-electrolyzed plating of nickel can also be carried out to form a uniform layer.

Still further, the protective layer 13 may be partially formed of carbon, and partially formed of aluminum. A protective metal layer is stronger than a carbon layer but can stand less bending. Thus, only a small portion of the metal protective layer 13 is formed of metal, and other portions are formed of carbon. According to experimentation, the portion formed of aluminum or nickel was able to bear bending to a radius of 5 mm. In the case of the transmission conduit 8 in which the protective layer 13 of aluminum of 1 to 3 microns and a quartz fiber 12 having a diameter of 125 micrometers, propagation time was almost the same as the case in which there is no protective layer 13. Even when the thickness of the protective layer 13 of aluminum was slightly uneven, the attenuation constant of about 2 dB/m at 1 MHz and about 10 dB/m at 20 MHz was realized, which is within a usable range. It is possible, by optimizing the thickness of the aluminum protective layer 13, to reduce the attenuation constant further. Since the impedance of aluminum to the ultrasonic wave is almost the same as that of a quartz fiber, attenuation is low. Although a uniform thickness of the quartz fiber 12 is described, depending on the frequency of the ultrasonic wave, it may be desirable to have a variable thickness. A thickness of the side of the vibrator 3 may be thicker because more energy is received, and the thickness on the tip side is thinner.

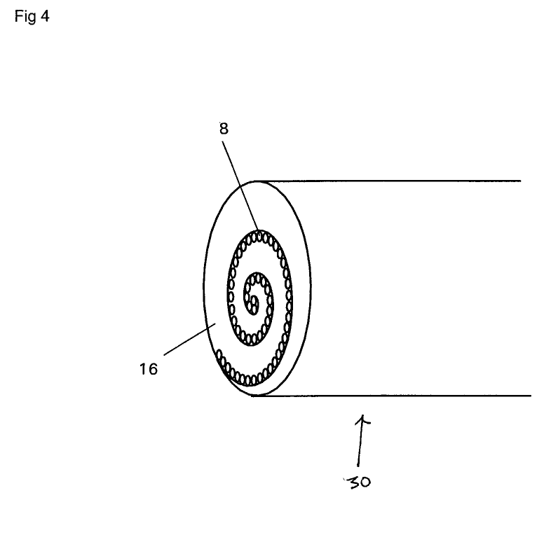

FIG. 4 is a perspective diagram of transmission unit 30 according to a second embodiment of the present invention, including a plurality of the transmission conduits 8 arranged in a bundle. A spacer 16 thickens gradually and a density of the transmission conduits 8 increases towards a center of the bundle.

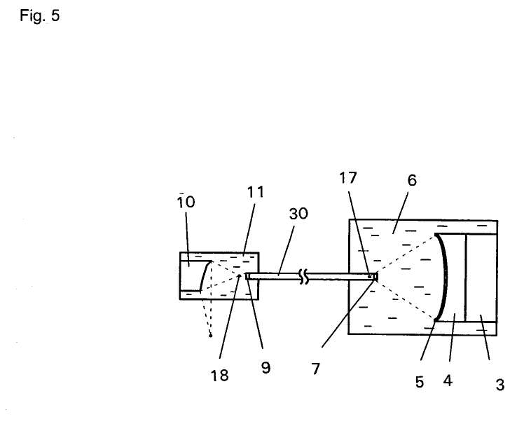

FIG. 5 illustrates the ultrasonic medical treatment/diagnostic equipment using the transmission unit 30 of FIG. 4. The focus 17 of the converged ultrasonic wave is displaced along an axis of the transmission unit 30. The ultrasonic wave emitted from the transmission unit 30 has a focus 18, which is displaced from the end of the transmission unit 30. Each of the transmission conduits 8 has the quartz fiber 12, with the protective layer 13, as in the first embodiment. However, since there is a plurality of the quartz fibers 12, the transmission unit 30 is thinner than in the first embodiment, and the bending of the transmission unit 30 is facilitated. Moreover, energy of the ultrasonic wave passing through the transmission unit 30 can be increased, thereby increasing the incineration power applied to the neoplasm.

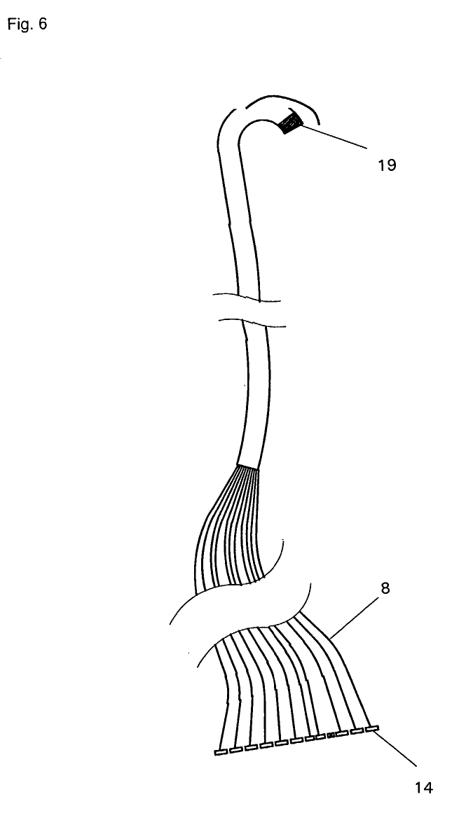

FIG. 6 illustrates a third embodiment of the present invention. While the first embodiment included a single dispatch element 14, this embodiment includes a plurality of the dispatch elements 14, each attached to a transmission conduit 8. The ends of the transmission conduits 8 come together to form a common adjustment layer 19. Due to the large number of dispatch elements 14, this embodiment can emit a powerful ultrasonic wave. Furthermore, since the transmission conduits 8 are arranged as a bundle, it is easy to bend the transmission conduits 8.

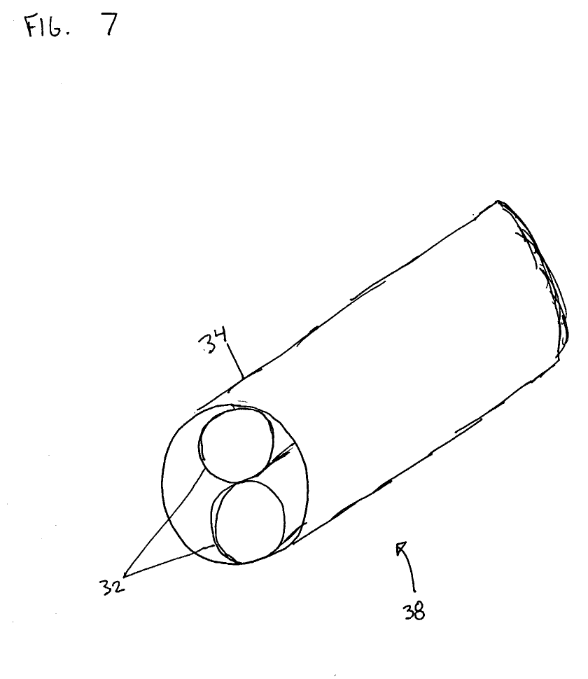

FIG. 7 shows a transmission conduit 38 according to a fourth embodiment the present invention. According to this embodiment, the quartz fibers 12 are inserted into holes of a honeycomb pipe formed by two or more stainless thin pipes 32 (0.13 mm inner diameter, 0.23 mm outer diameter) contained in a thick pipe 34 having a 5 mm diameter. To form this system, the quartz fibers 12 are inserted into two or more of the thin pipes 32. Next, the thin pipes 32 are bundled and inserted into the thick pipe 34, and both ends of each of the quartz fibers 12 are fixed, and ground down. Thus, the length of each of the quartz fibers 12 is made uniform.

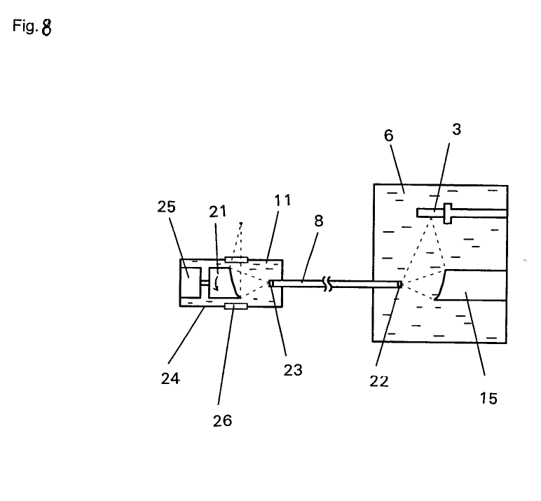

FIG. 8 is a schematic view showing the ultrasonic treatment/diagnostic equipment according to a fifth embodiment of the present invention, including a rotating sound mirror 21, which is rotated by motor 25. The ultrasonic wave which is emitted from the vibrator 3 is converged by a sound mirror 15, and passes through the transmission conduit 8 through an adjustment layer 22, and is sent to the tip part 24. In the tip part 24, the ultrasonic wave passes an adjustment layer 23 and is reflected by the sound mirror 21. Since the sound mirror 21 is rotated, the ultrasonic wave which is reflected and converged by the sound mirror 21 passes through an annular sound window 26, and hits the neoplasm or other affected part. The ultrasonic wave reflected from the affected part passes through the sound window 26 again, returns to the sound mirror 21, and is transmitted back through the transmission conduit 8. The ultrasonic wave further passes through the adjustment layer 22 and is reflected by the sound mirror 15, and converged on the vibrator 3. The vibrator 3 is made of a piezo-electric element of a titanium acid group, and generates voltage by vibration received from an exterior source (not shown). Therefore, if the voltage waveform which the vibrator 3 generates is indicated as an image, an image of the affected part can be made. Since the sound mirror 21 rotates, this embodiment obtains a picture of an affected part which surrounds the inserted tip part 24.

As described above, a potential advantage of the system is improved bending due to the reinforced glass fiber with the carbon protective layer. The use of carbon nanotube allows for bending to a small curvature, and therefore, the fiber can pass through curved pipelines, such as the urinary tract. Consequently, destruction of cancer cells and other undesirable growths can be performed without performing surgery.

Moreover, since the tip part 24 is a transmission-and-reception part inserted near the affected area, it is not necessary to irradiate portions of the body which have no pathological changes, as is the case when the ultrasonic wave is irradiated from the outside of the body.

Furthermore, according to one aspect thereof, the ultrasonic medical treatment equipment and ultrasonic diagnostic equipment may have a quartz fiber core inside of the transmission conduit. Since this core is prevented from touching the body, attenuation during transmission of the ultrasonic wave is small, and the strength of the ultrasonic wave is great.

Furthermore, by bundling cores, such as glass fibers, large amounts of energy can be transmitted to destroy kidney stones, or other large growths having large energy requirements. Moreover, since the glass fiber is thin, bending is easy.

When bundling the cores, phase differences between different fibers can be offset by changing the lengths of the fibers, thereby increasing efficiency. Furthermore, by rotating the mirror in the transmission-and-reception part, an ultrasonic wave can be transmitted circumferentially around the transmission-and-reception part, to obtain a complete image.

Still further, since the temperature of the part under medical treatment does not rise to the boiling point of water, a cancer cell will not scatter, as is the case with a high frequency scalpel, a laser surgery machine, or the external ultrasonic convergence medical treatment equipment.

Although a few preferred embodiments have been shown and described, it will be appreciated by those skilled in the art that changes may be made in these embodiments without departing from the principles and spirit of the invention, the scope of which is defined in the claims and their equivalents.

What is claimed is:

1. An ultrasonic medical treatment/diagnostic apparatus comprising:
a generating unit to generate an ultrasonic wave; and
a transmission unit to transmit the ultrasonic wave, comprising:
a fiber, and
a protective layer on a periphery of the fiber.

2. The ultrasonic medical treatment/diagnostic apparatus of claim 1, wherein the fiber comprises quartz.

3. The ultrasonic medical treatment/diagnostic apparatus of claim 1, wherein the protective layer comprises carbon.

4. The ultrasonic medical treatment/diagnostic apparatus of claim 1, wherein the protective layer comprises a carbon nano-tube.

5. The ultrasonic medical treatment/diagnostic apparatus of claim 1, wherein the protective layer comprises metal.

6. The ultrasonic medical treatment/diagnostic apparatus of claim 1, wherein the transmission unit further comprises:
a plurality of first pipes to receive a plurality of the fibers; and
a second pipe to receive the plurality of first pipes.

7. The ultrasonic medical treatment/diagnostic apparatus of claim 1, further comprising a plurality of the transmission units arranged in a bundle.

8. The ultrasonic medical treatment/diagnostic apparatus of claim 7, wherein a density of the transmission units in the bundle increases towards a center of the bundle.

9. The ultrasonic medical treatment/diagnostic apparatus of claim 1, wherein the ultrasonic wave transmitted by the transmission unit has a frequency of 1 MHz or greater.

10. The ultrasonic medical treatment/diagnostic apparatus of claim 1, wherein the transmission unit is bent to a radius of 5 mm.

11. The ultrasonic medical treatment/diagnostic apparatus of claim 7, further comprising a sound lens to converge the ultrasonic wave on the transmission unit,
wherein the ultrasonic wave is converged at a point axially displaced from an end of the transmission unit.

12. The ultrasonic medical treatment/diagnostic apparatus of claim 1, further comprising a sound lens to converge the ultrasonic wave on the transmission unit,
wherein the ultrasonic wave is converged at an end of the transmission unit.

13. The ultrasonic medical treatment/diagnostic apparatus of claim 1, further comprising:
a plurality of the transmission units; and
a plurality of dispatch units, respectively attached to each of the transmission units.

14. The ultrasonic medical treatment/diagnostic apparatus of claim 1, further comprising a sound mirror to receive the ultrasonic wave transmitted by the transmission unit.

15. The ultrasonic medical treatment/diagnostic apparatus of claim 14, wherein the sound mirror rotates.

16. The ultrasonic medical treatment/diagnostic apparatus of claim 1, further comprising:
a sound lens to converge the ultrasonic wave on the transmission unit; and
a silicone oil transmission medium to transmit the ultrasonic wave from the sound lens to the transmission unit.

17. The ultrasonic medical treatment/diagnostic apparatus of claim 9, wherein an attenuation constant of 2 dB/m at 1 MHz and 10 dB/m at 20 MHz is realized.

18. The ultrasonic medical treatment/diagnostic apparatus of claim 14, further comprising a tip part to enclose the sound mirror, wherein the tip part has a diameter of 2 mm or less.

19. The ultrasonic medical treatment/diagnostic apparatus of claim 1, wherein a thickness of the fiber increases in a direction of the generating unit.

20. The ultrasonic medical treatment/diagnostic apparatus of claim 18, wherein the tip part is a treatment/diagnostic part.

21. A transmission unit to transmit an ultrasonic wave in an ultrasonic medical treatment/diagnostic apparatus, the transmission unit comprising:
a fiber; and
a protective layer on a periphery of the fiber.

22. A method of treating/diagnosing a body, comprising:
inserting a transmission unit, comprising a fiber and a protective layer on a periphery of the fiber, inside the body;
generating an ultrasonic wave outside of the body; and
transmitting the ultrasonic wave inside the body via the transmission unit.

23. The method of claim 22, further comprising:
reflecting the transmitted ultrasonic wave to the body;
receiving a signal from the body in response to the reflected ultrasonic wave; and
generating an image from the received signal.

24. The method of claim 23, wherein the reflecting of the transmitted ultrasonic wave comprises rotating a reflecting unit.

25. The method of claim 24, wherein the generating of the image comprises generating an image of a portion of the body surrounding the reflecting unit.

26. The method of claim 24, further comprising:
inserting an endoscope into the body; and checking a position of the reflecting unit with the endoscope.
